(19) United States
(12) Patent Application Publication
(10) Pub. No.: US 2009/0208502 A1
(43) Pub. Date: Aug. 20, 2009

(54) APOPTOSIS-INDUCING PROTEIN COMPLEXES AND THERAPEUTIC USE THEREOF

(76) Inventor: Ralph Alexander Willemsen, Rotterdam (NL)

Correspondence Address: HOFFMANN & BARON, LLP, 6900 JERICHO TURNPIKE, SYOSSET, NY, 11791, US

(21) Appl. No.: 12/158,137

(22) PCT Filed: Dec. 20, 2005
(86) PCT No.: PCT/NL05/00878

§ 371 Date: Oct. 31, 2008

(51) Int. Cl. A61K 39/395; C07K 16/18; C12P 21/08; C12N 5/06

(52) U.S. Cl. 424/134.1; 530/387.3; 435/69.6; 435/375

(57) ABSTRACT

The invention relates to the fields of immunology and molecular medicine. In particular, it relates to protein complexes that can be applied as therapeutic agent to induce apoptotic cell death in a target cell population, for example tumour cells or virally infected cells. Provided is a multivalent monospecific protein complex comprising at least six polypeptides capable of recognizing and binding to a specific Major Histocompatibility Complex (MHC)-peptide complex, which complex induces apoptosis through the recognition of and binding to MHC-peptide molecules of a target cell. Also provided is the therapeutic use of a protein complex, for example for the manufacture of a medicament for the treatment of cancer, a viral or a microbial infection.

The invention relates to the fields of immunology and molecular medicine. In particular, it relates to protein complexes that can be applied as therapeutic agent to induce apoptotic cell death in a target cell population, for example tumour cells or virally infected cells. In particular, it relates to multivalent protein complexes capable of inducing apoptosis through the recognition of and binding to tumour- or virus-derived peptides presented by Major Histocompatability Complex (MHC) molecules of a target cell.

The primary immunological function of MHC molecules is to bind and “present” antigenic peptides to form a MHC-peptide (MHC-p) complex on the surface of cells for recognition and binding by antigen-specific T cell receptors (TCRs) of lymphocytes. With regard to their function, two classes of MHC-peptide complexes can be distinguished (Germain, R., Cell 76 (1994) 287-299): (i) MHC class I-peptide complexes can be expressed by almost all nucleated cells in order to attract CD8⁺ cytotoxic T cells, and (ii) MHC class II-peptide complexes are constitutively expressed only on so-called antigen presenting cells (APCs), such as B lymphocytes, macrophages or dendritic cells (DCs). MHC class I molecules are composed of a variable heavy chain, invariable β microglobulin and antigenic peptide. The MHC class II molecules are characterized by distinctive α and β polypeptide subunits that combine to form αβ heterodimers characteristic of mature MHC class II molecules. Differential structural properties of MHC class I and class II molecules account for their respective roles in activating different populations of T lymphocytes. Cytotoxic T_{C} lymphocytes (CTLs) bind antigenic peptides presented by MHC class I molecules. Helper T_{H} lymphocytes bind antigenic peptides presented by MHC class II molecules. MHC class I and class II molecules differentially bind CD8 and CD4 cell adhesion molecules. MHC class I molecules specifically bind CD8 molecules expressed on cytotoxic T_{C} lymphocytes, whereas MHC class II molecules specifically bind CD4 molecules expressed on helper T_{H} lymphocytes.

The sizes of the antigenic peptide-binding pockets of MHC class I and II molecules differ; class I molecules bind smaller antigenic peptides, 8-10 amino acid residues in length, whereas class II molecules bind larger antigenic peptides, 13-18 amino acid residues in length.

In humans, MHC molecules are termed human leukocyte antigens (HLA). HLA-associated peptides are short, encompassing 9-25 amino acids (Kropshofer, H. & Vogt, A. B., Immunol Today 18 (1997) 77-82). Humans synthesize three different types of class I molecules designated HLA-A, HLA-B, and HLA-C. Human class II molecules are designated HLA-D, e.g. HLA-DR. It has been shown in the art that antibodies against MHC class I and class II molecules can induce apoptosis in cells expressing said MHC molecules. Wallen-Ohman et al. reported that ligation of MHC class I by murine monoclonal antibody (mAb) induces apoptosis in human pre-B cell lines, in promyelocytic cell lines and in CD40-stimulated mature B cells (Int Immunol. 1997; 9(4):599-606). Both mouse and human anti-HLA class I antibodies were shown to have apoptosis-inducing effects on human lymphocytes (Genestier et al., Blood. 1997, 15; 90(2):726-35; Daniel et al., Transplantation. 2003 Apr. 27; 75(8):1380-6). Newell et al. reported that ligation of MHC class II molecules with anti-class II antibodies mediates apoptotic cell death in resting B lymphocytes (Proc Natl Acad Sci USA. 1993 Nov. 15; 90(22):10459-63). HLA-DR specific monoclonal antibodies have been described that can induce apoptosis of HLA-DR positive cells (Vidovic et al. Cancer Lett. 1998, 19; 128(2):127-35; see also U.S. Pat. No. 6,416,958).

Thus, it is known that binding of MHC class I or II molecules by several anti-MHC antibodies can have an apoptosis-inducing effect. However, the therapeutic application of the currently available anti-MHC antibodies has been hampered by the lack of target cell specificity. Since the known antibodies are directed primarily against an epitope of the MHC molecule itself (e.g. HLA-DR), it is the cell surface expression of said MHC epitope which determines whether or not a cell can be triggered to undergo apoptosis. Because MHC class I and II molecules are expressed on both normal and diseased cells, it is clear that currently available antibodies cannot discriminate between normal and abnormal (e.g. diseased) cells. As a consequence, their therapeutic value is significantly reduced by the side-effects caused by unwanted apoptosis of healthy cells. Furthermore, methods to induce apoptosis via MHC class I or II are strictly dependent on external cross-linking of anti-MHC antibodies.

It is a goal of the present invention to overcome the above limitations and provide a therapeutic agent that allows for the induction of apoptosis with an improved specificity. In particular, it is a goal to selectively induce apoptosis of a cell population of interest, for example of tumour cells expressing a tumour antigen or virally infected cells expressing a viral antigen, while minimizing or totally avoiding the loss of viability of healthy cells.

These goals are met by the surprising finding that a multivalent protein complex comprising multiple antigen-specific, MHC-restricted T cell receptors (TCRs) and/or MHC-restricted antibodies can efficiently induce apoptosis in a population of only those target cells which express the antigen. The killing was found to be strictly dependent on the presence of the relevant antigen in an MHC context. This finding opens up the possibility to selectively kill a population of cells that are positive for a certain MHC-peptide complex of interest, for example tumour cells expressing HLA class I molecules complexed with peptides derived from tumour-associated antigens.

Without wishing to be bound by theory, it is thought that a multivalent, monospecific protein complex of the invention induces apoptosis via the clustering of a number of identical MHC-p complexes on the cell surface of a target cell. The data shown herein suggest that clustering of three MHC-p complexes is not sufficient for apoptosis induction, whereas a hexavalent complex is very efficient in inducing apoptosis. Thus, apoptosis induction requires the binding of at least four, preferably at least five, more preferably at least six MHC-p complexes by one multivalent, monospecific protein complex. In one embodiment, the complex consists of four, five, six, seven, eight, nine, ten, eleven or twelve polypeptides, each polypeptide capable of recognizing and binding to a specific Major Histocompatibility Complex (MHC)-peptide complex. In contrast to the known methods for apoptosis induction using anti-MHC antibodies, a multivalent protein complex disclosed herein can induce apoptosis itself and does not require any external cross-linking.

The invention therefore relates to a multivalent monospecific protein complex comprising at least six polypeptides capable of recognizing and binding to a specific Major Histocompatibility Complex (MHC)-peptide complex. Said at least six polypeptides recognize the same MHC-peptide (MHC-p) complex, i.e. the multivalent protein complex is monospecific with respect to the MHC-p complex. The polypeptide which specifically recognizes and binds to a MHC-p complex can be a TCR or a functional fragment thereof (together herein referred to as TCRs) and/or an antibody which mimics TCR specificity, for example a genetically engineered antibody such as a single-chain variable fragment (sc-Fv). Also, a multivalent complex of the invention may contain TCRs as well as MHC-restricted antibodies, provided that both types of polypeptides recognize the same peptide antigen.

Multivalent TCR complexes and therapeutic applications thereof are known in the art. WO2004/050705 in the name of Avidex Ltd. discloses a multivalent TCR complex comprising at least two TCRs, linked by a non-peptidic polymer chain or a peptidic linker sequence. The TCR complex may be used for targeted cell delivery of therapeutic agents, such as cytotoxic drugs, which can be attached to the TCR complex. Di-, tri- and tetravalent TCR complexes are disclosed but divalent TCR complexes are preferred. Importantly, complexes of more than four TCRs are not described. Furthermore, WO2004/050705 focuses solely on the use of a multivalent TCR complex for the delivery of a therapeutic agent, e.g. a toxic moiety for cell killing, to a target cell. It does not teach or suggest the apoptosis-inducing capacity of a multivalent TCR complex itself. The antigen-specific, MHC-restricted binding capacity of a polypeptide complex of the invention is sufficient to induce apoptosis of a target cell expressing the relevant antigen. The complex may therefore be “bare” i.e. devoid of any additional or attached cytotoxic agent or toxic moiety as for example is required in WO2004/050705. For therapeutic application of a multivalent protein complex of the invention, it is preferred that the size of the complex is small enough to allow entry in the blood stream. Preferably it can penetrate the tissue which comprises a target cell population, for instance tumour tissue, especially poorly vascularized solid tumors. Therefore, the molecular weight of a multivalent complex is preferably less than about 400 kDa, more preferably less than about 300 kDa, like 200, 250, 270 or 290 kDa.

The polypeptides within a complex of the invention, be it antigen-specific MHC-restricted TCRs, TCR-like antibodies or combinations thereof, can be linked or connected to each other in any suitable manner. In one embodiment, the individual polypeptides are covalently attached to each other, either directly or indirectly. For example they can be connected by chemical cross-linking or via a non-peptidic polymer chain (see WO2004/050705). Methods for chemical coupling of polypeptides via functional coupling sites (e.g. SH groups) are well known in the art.

In another embodiment, the polypeptides are non-covalently connected to each other. In one aspect, multiple polypeptides are linked via a linker peptide capable of binding two or more polypeptides. The linker peptide may comprise two or more, like three, four, five or six, specific binding sites for the polypeptide. The polypeptide may comprise a binding ligand for the binding site on the linker peptide. In one embodiment, each of the polypeptides within a multivalent complex comprises a binding ligand that allows for non-covalent binding of the polypeptide to a linker peptide. The binding ligands of each of the polypeptides can be the same or they can be different. Polypeptides having different binding ligands allow for influencing the spatial arrangement of the polypeptides when bound to a linker peptide.

In a preferred embodiment, a linker peptide comprises a multimerisation motif via which the linker peptide can multimerize with another linker peptide comprising said motif. Multimerisation of linker peptides, each linker peptide capable of binding at least one MHC-p specific polypeptides, is very suitable for the assembly of multiple polypeptides into one complex. Multimerisation motifs include dimerization, trimerisation, tetramerization, pentamerization and hexamerization motifs. Exemplary multivalent protein complexes consist of the following components: six polypeptides bound to one linker peptide with six polypeptide binding sites (hexavalent complex); two linker peptides, each comprising a dimerization motif and three polypeptide binding sites (hexavalent); two linker peptides, each with a dimerization motif and four polypeptide binding sites (octavalent); three linker peptides, each with a trimerisation motif and two or three polypeptide binding sites (hexa- or nonavalent); two linker peptides, each with a tetramerization motif and two polypeptide binding sites (octavalent); and so on.

The size limitation mentioned above can pose some practical restrictions regarding a) the number of polypeptides that are assembled into one multivalent complex i.e. the valency of the complex, as well as regarding b) the manner in which they are assembled since one typically wants to minimize the size and weight of components that do not contribute to the actual MHC-p binding on a target cell. Therefore, protein complexes with a valency up to nine are preferred. Furthermore, the use of linker peptides having a multimerisation motif allows for the assembly of the polypeptides into a relatively compact complex as compared to a single linker peptide or non-peptidic linker to which all polypeptides are attached. It is also possible to fuse a linker peptide to a polypeptide.

Binding ligands that can be used to link a polypeptide to a binding site on a linker peptide are known in the art. Any binding ligand, be it of peptidic or non-peptidic origin, can be used as long as there is a binding site available which can be part of a linker peptide. Preferably, a polypeptide contains a single binding ligand in order to prevent the binding of one polypeptide to more than one linker peptide, which could result in the formation of unwanted “chains” consisting of alternating linker peptides and polypeptides instead of the desired protein complex. The binding ligand may be covalently or non-covalently attached to the polypeptide. Covalent attachment is preferred. This can for example be achieved by chemical coupling of the binding ligand to the polypeptide or by genetic fusion. Preferably, the binding ligand is a peptide whose encoding nucleic acid sequence can be genetically fused to the nucleic acid sequence encoding the MHC-p-specific polypeptide. More preferably, both the binding ligand and the binding site are of peptidic nature such that they can be genetically fused to a polypeptide and a linker peptide, respectively. The fusion of the binding ligand to the polypeptide is typically performed C-terminal from the polypeptide, but may also be N-terminal. As will be discussed below, the position of one or more binding sites within the linker peptide can vary.

Suitable binding ligand/binding site pairs that can be used to bind one or more MHC-p-specific polypeptides to a linker peptide include the biotin/(strept)avidin pair and dimerization domains, such as leucine zippers. The biotin-streptavidin system is the strongest noncovalent biological interaction known, having a dissociation constant, K(d), in the order of 4×10(−14) M. The strength and specificity of the interaction has led it to be one of the most widely used affinity pairs in molecular, immunological, and cellular assays. dimerization domain, such as a leucine zipper domain. In one embodiment, use is made of an S—S zipper (De Kruif J, Logtenberg T. J Biol Chem. 1996 Mar. 29; 271(13):7630-4. In a preferred embodiment, the small polypeptide neurotoxin alpha-bungarotoxin (BTX) is used as binding ligand. Because BTX can bind with high affinity to a 13-aa alpha-bungarotoxin (BTX)-binding site (BBS) (Harel et al., (2001) Neuron 32, 265-275), a BTX-tagged polypeptide can be non-covalenty bound to a linker peptide comprising a BTX.

Multimerization motifs for use in a linker peptide can be found in naturally occurring proteins in both prokaryotes and eukaryotes. The biotin/streptavidin system has previously been used to produce TCR tetramers for in vitro binding studies. However, streptavidin is a microbially-derived polypeptide and as such not ideally suited to use in a therapeutic complex. Other examples of multimerization motifs include the trimerisation signal of bacteriophage T4 fibritin (Efimov et al., (1994) J. Mol. Biol. 242, 470-486., the Neck Region Peptide (NRP) of human Lung Surfactant D protein (trimerisation motif; see Hoppe et al., (1994) FEBS Lett. 344:191-195), and the modified leucine zipper trimerisation domain (Harbury et al., (1993) Science 262, 1401-1407).

For therapeutic applications, it is preferred that the motif is not derived from a pathogenic organism. More preferably, a mammalian multimerization motif is used to assemble a protein complex of the invention. Human multimerization motifs are most preferred. There are a number of human proteins that contain a multimerisation domain that can be used in the production of a multivalent complex of the invention. For example the tetramerisation domain of p53 which has been used to produce tetramers of scFv antibody fragments can be used. Haemoglobin also has a tetramerization motif that could be of use in the present invention. In one preferred embodiment, the trimerisation motif NRP of human Lung Surfactant D protein is used.

The invention thus also provides a linker peptide for use in a complex of the invention. The linker peptide comprises at least one binding site for a polypeptide comprising a binding ligand for said binding site. Preferably, the linker peptide comprises two or more of such binding sites. For reasons described above it is also preferred that the linker peptide comprises a multimerization motif. The binding sites and multimerization motif can be present within the linker peptide as separate or as joined segments, i.e. they can be spaced by a small stretch of amino acid residues, like 1-50, preferably 1-20, more preferably 1-10 amino acid residues. The order in which the segments are arranged can vary. The spacing between the multimerization motif and a binding site should be sufficient to allow for a) the multimerization between the linker peptide and another linker peptide, and b) the binding of a polypeptide to a binding site. It is preferred that a multimerization motif is flanked on each side by one or more binding sites. In one embodiment, a linker peptide comprises from N to C terminus the following segments: binding site (e.g. BBS)-multimerization motif (e.g. NRP)-binding site (e.g. BBS). A linker peptide may furthermore comprise stretches of amino acids which aid in the expression and/or secretion of the linker peptide, in particular if the linker peptide is produced by a recombinant host cell. For example, it may contain an N-terminal secretion signal sequence to promote secretion of the peptide in the medium. A suitable secretion signal is the interleukin-2 (IL-2) secretion signal sequence. Other useful sequences include those that allow for convenient protein purification, in particular affinity tags known in the art such as c-myc-tag, 6×His-tag, HA-tag, and the like. One linker peptide may comprise one or more of such tags, optionally flanked on one or both sides by a short flexible linker sequence (e.g. alternating Gly and Ser residues). In one embodiment, the invention provides a linker peptide comprising the following segments (from N- to C-terminus): secretory signal sequence-binding site-linker-affinity tag-linker-multimerization motif-linker-affinity tag-linker-binding site. An exemplary linker peptide of this type is the Hexa-Tag peptide described in the Examples.

Also provided herein is a nucleic acid encoding a linker peptide of the invention. As is described in the Examples, standard recombinant DNA technology can be used to construct the nucleic acid.

According to the invention, any polypeptide capable of recognizing and binding to a specific MHC-peptide complex, class I or II, is suitably used in a multivalent apoptosis-inducing protein complex. In one embodiment, the complex comprises at least one polypeptide, preferably at least two, more preferably at least four, like six or even more polypeptides, comprising amino acid sequences corresponding to extracellular constant (C) and variable (V) region sequences of a native TCR. In the complexes of the invention, the TCR molecules may be single chain T cell receptor (scTCR) polypeptides or two-chain (dimeric) TCR (tcTCR) polypeptide pairs. scTCR polypeptide, or tcTCR polypeptide pairs may be constituted by TCR amino acid sequences corresponding to TCR extracellular constant and variable region sequences, with a variable region sequence of the scTCR corresponding to a variable region sequence of one TCR chain being linked by a linker sequence to a constant region sequence corresponding to a constant region sequence of another TCR chain; the variable region sequences of the tcTCR polypeptide pair or scTCR polypeptide are mutually orientated substantially as in native TCRs; and in the case of the scTCR polypeptide a disulfide bond which has no equivalent in native T cell receptors links residues of the polypeptide.

In one embodiment, at least one polypeptide is a single-chain T cell receptor (scTCR) polypeptide, for example an scTCR comprising the variable (V) region of an antigen-specific TCR, optionally further comprising an extracellular constant (C) region of an antigen-specific TCR. In another embodiment, at least one polypeptide is a two-chain TCR (tcTCR) comprising the extracellular variable (V) and constant (C) regions of an antigen-specific TCR. Said scTCR or tcTCR for example comprise the α and β chains pair or the γ and δ chain pair of an antigen-specific TCR.

For αβ-analogue scTCRs or tcTCRs present in the complexes of the invention, the requirement that the variable region sequences of the α and β segments are mutually orientated substantially as in native αβ TCRs is tested by confirming that the molecule binds to the relevant TCR ligand (pMHC complex)—if it binds, then the requirement is met. Interactions between a polypeptide, be it a TCR or an antibody-based polypeptide, and pMHC complexes can be measured using a BIAcore3000™ or BIAcore 2000™ instrument. WO99/6120 provides detailed descriptions of the methods required to analyse TCR binding to MHC-peptide complexes. In the case of γδ-analogue TCRs present in the complexes of the invention the cognate ligands for these molecules are unknown and therefore secondary means of verifying the conformation of these molecules such as recognition by antibodies can be employed. The monoclonal antibody MCA991T (available from Serotec), specific for the δ chain variable region, is an example of an antibody appropriate for this task.

scTCRs are artificial constructs consisting of a single amino acid strand, which like native heterodimeric TCRs bind to MHC-peptide complexes. In one embodiment, a polypeptide encodes a two-domain (2D) scTCR comprising the extracellular variable (V) region VαVβ chains of the TCR linked by a linker. In another embodiment, the polypeptide comprises a three-domain (3D) scTCR comprising the extracellular variable (V) and constant (C) regions of the TCR, for example a scTCR comprising the VαVβCβ or VαVβCα chains of an antigen-specific TCR. The linker that links the VαVβ can be selected from standard linkers known in the art such as oligopeptides of 15-20 amino acids that allow for flexibility and proper association between the two V domains (see also the teaching of WO2004/033685). scTCR polypeptides present in the complexes of the invention can be those which have, for example, a first segment constituted by an amino acid sequence corresponding to a TCR α or δ chain variable region sequence fused to the N terminus of an amino acid sequence corresponding to a TCR α chain constant region extracellular sequence, a second segment constituted by an amino acid sequence corresponding to a TCR β or γ chain variable region fused to the N terminus of an amino acid sequence corresponding to TCRβ chain constant region extracellular sequence, a linker sequence linking the C terminus of the first segment to the N terminus of the second segment, or vice versa, and a disulfide bond between the first and second chains, said disulfide bond being one which has no equivalent in native αβ or γδ T cell receptors, the length of the linker sequence and the position of the disulfide bond being such that the variable region sequences of the first and second segments are mutually orientated substantially as in native αβ or δγ TCRs. Such polypeptides are e.g. described in WO2004/033685.

Two-chain TCRs (tcTCRs) in a complex of the invention can be those which are constituted by a first polypeptide wherein a sequence corresponding to a TCR α or δ chain variable region sequence is fused to the N terminus of a sequence corresponding to a TCR α chain constant region extracellular sequence, and a second polypeptide wherein a sequence corresponding to a TCR β or γ chain variable region sequence fused to the N terminus of a sequence corresponding to a TCR P chain constant region extracellular sequence. The first and second polypeptides can be linked to form an MHC-p-specific polypeptide of the invention by a disulfide bond which has or has no equivalent in native αβ or γδ TCRs. In one embodiment, a polypeptide is a two-chain TCR comprising the extracellular V and C regions, such as a tcTCR comprising the VαCα+VβCβ chains of the TCR.

The constant region extracellular sequences present in the above scTCRs and tcTCRs preferably correspond to those of a human TCR, as do the variable sequences. However, the correspondence between such sequences does not need to be 1:1 on the amino acid level. N- and/or C-truncation, and/or amino acid deletion and/or substitution relative to the human TCR sequences is acceptable, provided that the overall result is mutual orientation of the α and β variable region sequences, or γ and δ variable region sequences as in native αβ, or γδ TCRs, respectively such that MHC-binding capacity is maintained. In particular, because the constant region extracellular sequences are not directly involved in contacts with the ligand (MHC-p complex) to which the scTCR or tcTCR binds, they may be shorter than, or may contain substitutions or deletions relative to, extracellular constant domain sequences of native TCRs.

Alternative or in addition to a TCR polypeptide, an MHC-p specific polypeptide in a complex of the invention is an MHC-restricted, antigen-specific TCR-like antibody (Ab) or functional fragment thereof. Protein fragments consisting of the minimal binding subunit of antibodies known as single-chain antibodies (scFvs) have excellent binding specificity and affinity for their ligands. In contrast to antibodies, scFvs lack the non-binding regions, can be selected in the company of competing antigens, and therefore have potential for higher specificity/sensitivity. For example, the polypeptide is a single-chain Ig (scFv-Ig) comprising the variable (V) region and, optionally, the extracellular constant (C) region of an antibody specifically reactive with an antigen of interest in a MHC context, for instance a virus- or tumour antigen specific antibody. The TCR-like Ab polypeptide can also be a two-chain antibody fragment, e.g. comprising the extracellular V and C regions of an antibody.

Antigen-specific antibodies or functional fragments thereof can be provided by standard procedures known in the art, including classical immunization procedures and genetic engineering. Of particular interest is the use of phage display technology. Many reviews on phage display are available, see for example Smith and Petrenko [1997] Chem. Rev. 97:391-410. Briefly, phage display technology is a selection technique in which a library of variants of a peptide or human single-chain Fv antibody is expressed on the outside of a phage virion, while the genetic material encoding each variant resides on the inside. This creates a physical linkage between each variant protein sequence and the DNA encoding it, which allows rapid partitioning based on binding affinity to a given target molecule by an in vitro selection process called panning. In the present invention, the target molecule is for example a recombinant MHC-peptide complex of interest, such as melanoma-associated antigen (MAGE)-A1 presented by HLA-A1 molecules. In its simplest form, panning is carried out by incubating a library of phage-displayed peptides with a plate (or bead) coated with the target (i.e. MHC-p of interest), washing away the unbound phage, and eluting the specifically bound phage. The eluted phage is then amplified and taken through additional binding/amplification cycles to enrich the pool in favour of binding sequences. After 3-4 rounds, individual clones are typically characterized by DNA sequencing and ELISA. The DNA contained within the desired phage encoding the particular peptide sequence can then be used as nucleic acid encoding an antibody-based polypeptide for use in a multivalent apoptosis-inducing complex of the invention.

The invention is primarily exemplified by the generation of a multivalent protein complex which is specific for a tumour- or viral antigen. These complexes have therapeutic value in the treatment of cancer and viral infections. However, the skilled person will appreciate that the present invention is not limited to any type of antigen, and that complexes are provided which can selectively kill target cells expressing any antigen, known or still to be discovered.

Preferably, a polypeptide is capable of recognizing and binding to a viral epitope, a cancer-specific epitope or an epitope associated with autoimmune disorders. The epitope is for example selected from the group consisting of HTLV-1 epitopes, HIV epitopes, EBV epitopes, CMV epitopes and melanoma epitopes. In a preferred embodiment, a multivalent complex comprises at least six polypeptides capable of recognizing and binding to an MHC class I or class II-tumour antigen complex, in particular melanoma associated antigens. Human tumor antigens presented by MHC class II molecules have been described, with nearly all of them being associated to malignant melanoma. The first melanoma antigenic peptide found was MAGE-1 (Traversari et al. J Exp Med. 1992 Nov. 1; 176(5):1453-7) Furthermore, 3 melanoma epitopes were found to originate from the MAGE family of proteins and presented by HLA-DR11 and HLA-DR13 (Manici S et al., J Exp Med 1999; 189, 871-876). Another set of melanoma antigens, known to contain also MHC class I tumor antigens, comprises Melan-A/MART-1 (Zarour H M et al., PNAS 2000; 97, 400-405), gp100 and annexin II (Li K et al. Cancer Immunol Immunother 1998; 47, 32-38). For an overview of T cell epitopes that are use of use for the present invention, also see www.cancerimmunity.org/peptidedatabase/Tcellepitopes.htm.

A further aspect of the invention relates to method for providing a protein complex according to the invention. As described herein above, it typically involves providing a nucleic acid encoding the desired polypeptide(s) which make up the complex, and, if the polypeptides are to be attached non-covalently, optionally also a construct encoding a linker peptide. Said nucleic acid construct(s) can be introduced, preferably via a plasmid or expression vector, into a host cell capable of expressing the construct(s). In one embodiment, a method of the invention to provide a multivalent apoptosis-inducing protein complex comprises the steps of providing a host cell with one or more nucleic acid(s) encoding said at least six polypeptides capable of recognizing and binding to a specific Major Histocompatibility Complex (MHC)-peptide complex and, optionally, a nucleic acid encoding a linker peptide and allowing the expression of said nucleic acids by said host cell. Preferred host cells are mammalian host cells, more preferably human host cells. Suitable host cells include human embryonic kidney (HEK-293T) or Chinese hamster ovary (CHO) cells, which can be commercially obtained. Insect cells, such as S2 or S9 cells, may also be used using baculovirus or insect cell expression vectors. The polypeptides produced (either the TCRs and/or the linker peptides) can be extracted or isolated from the host cell or, if they are secreted, from the culture medium of the host cell. Thereafter they can be assembled in vitro into a multivalent protein complex. If all components of a complex are produced by the same host cell, they may “self-assemble” into a protein complex such that isolation of the individual components may not be necessary. Thus, in one embodiment a method of the invention comprises providing a host cell with one or more nucleic acid(s) encoding said at least six polypeptides capable of recognizing and binding to a specific Major Histocompatibility Complex (MHC)-peptide complex and at least a linker peptide, allowing the expression of said nucleic acids by said host cell; and assembling the resulting peptides into the protein complex, wherein said polypeptides and linker peptides are secreted and assembled in the culture medium of said host cell. Methods for the recombinant expression of (mammalian) proteins in a (mammalian) host cell are well known in the art. The constructs can be introduced sequentially or simultaneously in a host cell. It is also possible to produce the TCR-(like) polypeptides in a host cell and attach the purified polypeptides to each other by chemical cross-linking.

As will be clear, a protein complex of the invention finds its use in many therapeutic and non-therapeutic, e.g. scientific, applications. Provided herein is a method for inducing ex vivo or in vivo apoptosis of a target cell, comprising contacting said cell with a protein complex according to the invention an amount that is effective to induce apoptosis. The target cells can be conveniently contacted with the culture medium of a host cell that is used for the recombinant production of the components (polypeptides, linker peptides) constituting the protein complex. In one embodiment, it can be used for in vitro apoptosis studies, for instance studies directed at the elucidation of molecular pathways involved in MHC class I and II induced apoptosis . . . . Complexes of the invention may also be used for the detection of (circulating) tumor cell or virally infected cells, for the target-cell specific delivery of cytotoxic compounds or the delivery of immune-stimulatory molecules.

Preferably, the protein complex is used for triggering apoptosis of diseased cells in a subject, more preferably a human subject. For therapeutic applications in humans it is of course preferred that a protein complex does not contain peptides of non-mammalian origin. More preferred are protein complexes which only contain human peptide sequences. It is demonstrated herein that a method of the invention allows for the killing of cells in an antigen-specific, MHC-restricted fashion. Therefore, a therapeutically effective amount of a protein complex capable of recognizing and binding to a disease-specific epitope can be administered to a patient to stimulate apoptosis of diseased cells expressing the epitope without affecting the viability of (normal) cells not expressing said disease-specific epitope. In a specific embodiment, the disease-specific epitope is a cancer-epitope, for example a melanoma-specific epitope. The killing of diseased cells while minimizing or even totally avoiding the death of normal cells will generally improve the therapeutic outcome of a patient following administration of the protein complex.

Accordingly, there is also provided a protein complex according to the invention as medicament. In another aspect, the invention provides the use of a protein complex for the manufacture of a medicament for the treatment of cancer, a viral or microbial infection, autoimmune disease or any other disease of which the symptoms are reduced upon killing the cells expressing a disease-specific antigen or epitope. For example, a protein complex is advantageously used for the manufacture of a medicament for the treatment of melanoma.

Provided as well is a pharmaceutical composition comprising as an active ingredient a protein complex according to the invention and a pharmaceutically acceptable carrier. The pharmaceutical composition may of course contain one or more additional active ingredients that are commonly used for the treatment of a given disease. A protein complex may be administered by various routes to a subject in need thereof. It can be administered intravenously (IV) or parenterally.

### FIGURE LEGENDS

FIG. 1: Apoptosis-induction of target cells by a multivalent MHC-restricted, antigen-specific protein complex.

FAM-VAD-FMC staining of MZ2-MEL3.0 cells that were incubated with:

Panel (A) Mock supernatant derived from 293T transfected with empty pBullet vector (10 μg).

Panel (B) Tri-TAG-scFv Hyb3 supernatant derived from 293T cells transfected with the pBullet-tri-tag and pBullet scFv Hyb3/BTX (5 μg each).

Panel (C) Hexa-TAG-scFv Hyb3 supernatant derived from 293T cells transfected with the pBullet hexa-tag and pBullet scFv Hyb3/BTX (5 μg each). Cells were incubated for 4 hours with supernatant and analysed by the caspatag assay (Intergen). Shown are cells stained with FAM-VAD-FMC.

FIG. 2: HLA-A1/MAGE-A1 specific induction of apoptosis by Hexa-TAG scFv Hyb3. For details see Item 4.2 of the Experimental Section.

FIG. 3: Hexa-TAG-scTCR MPD induces HLA-A2/gp100 specific apoptosis. For details see Item 4.3 of the Experimental Section.

### EXPERIMENTAL SECTION

The invention is exemplified by the Examples below.

### Materials and Methods

### Cells

Target cell lines used in this study are: (i) the HLA-A1^{POS}, MAGE-1^{POS} melanoma cell line MZ2-MEL.3.0, (ii) the HLA-A1^{POS}, MAGE-1^{NEG} melanoma cell line MZ2-MEL 2.2 (kindly provided by T. Boon and P. Coulie, Brussels, Belgium), (iii) the HLA-A1^{POS} EBV transformed B cell blasts APD, (iv) the HLA-A2^{POS}, gp100^{POS} melanoma cell line BLM gp100, (v) the HLA-A2^{POS}, gp100^{NEG} melanoma cell line BLM, and (vi) the HLA-A2^{POS} EBV transformed B cell blasts BLM. The human embryonic kidney cell line 293T (kindly provided by Y. Soneoka, Oxford, UK) was used as cell line for the production of scTCR and scFv complexes.

### Immunofluorescence Analysis of Apoptotic Human Cells.

### Caspase 3

Caspase 3 activity in apoptotic cells was determined by the caspaTag, caspase activity kit from Intergen (Intergen, Purchase, N.Y., USA). Briefly, 1×10⁶ cells were incubated with the Caspase-3 inhibitor FAM-VAD-FMC for 30 min, followed by 2 wash steps. Cells were fixed and analysed on a FACSCAN instrument (Becton Dickinson Biosciences, San Jose, USA).

### Annexin V/7-AAD Staining

Apoptosis was determined by double staining using Annexin V (BD-Pharmingen) and 7-AAD (Sigma). Briefly, Cells (1×10⁶) were harvested, washed with PBS and resuspended in 0.5 ml Annexin V binding buffer (2.5 mM CaCl₂.). After addition of Annexin V and 7-AAD (0.2 μg/total) cells are incubated for 30 min at 4° C., washed with PBS and analysed on a FACSCAN instrument (Becton Dickinson Biosciences, San Jose, USA).

### EXAMPLES

### 1.: Construction of scFv Hyb3 and scTCR MPD Polypeptides

1.1: Isolation and Cloning of a MAGE-1 Specific Single-Chain Variable Fragment (scFv) from a Large Phage Display Library.

Standard cloning techniques were used in the examples below. Techniques are described in: Molecular Cloning; A Laboratory Manual (Cold Spring Harbor Press, Cold Spring Harbor, N.Y.) by Maniatis et al.

Selection of a Human Antibody Fragment Directed Against the Tumor T-Cell Epitope HLA-A1-MAGE-A1 from a Nonimmunized Phage-Fab Library.

To obtain a human antibody directed against a peptide antigen encoded by gene melanoma-associated antigen (MAGE)-A1 and presented by HLA-A1 (human class MHC class I) molecules, a large phage Fab antibody repertoire was used for selection on a recombinant version of the complex HLA-A1-MAGE-A1 produced by in vitro refolding, essentially as described in “Direct selection of a human antibody fragment directed against the tumor T-cell epitope HLA-A1-MAGE-A1 from a nonimmunized phage-Fab library” by Chames et al., Proc Natl Acad Sci USA. 2000 Jul. 5; 97(14):7969-74.

In brief:

Selection of Phage-Antibodies on Biotinylated Complexes. A large human Fab library containing 3.7×10¹⁰ antibody fragments was used for the selection. Phages (10¹³) were first preincubated 1 h at room temperature in 2% nonfat dry milk-PBS in an immunotube coated with streptavidin (10 μg/ml) to deplete for streptavidin binders. Streptavidin-coated paramagnetic beads (200 μl; Dynal, Oslo) were also incubated in 2% milk-PBS for 1 h at room temperature. Phages were subsequently incubated for 1 h with decreasing amounts of biotinylated complexes (500, 100, 20, and 4 nM for rounds 1-4, respectively). Streptavidin beads were added, and the mixture was left for 15 min on a rotating wheel. After 15 washes with 0.1% Tween-PBS, bound phages were eluted by a 10-min incubation with 60 μl of 50 mM DTT, thus breaking the disulfide bond in between the complex and the biotin. The eluted phages were diluted in PBS to 1 ml and 0.5 ml were used to infect E. coli strain TG1 cells grown to the logarithmic phase (OD₆₀₀ of 0.5). The infected cells were plated for amplification. After infection of TG1 cells for 30 min at 37° C., bacteria were grown overnight at 30° C. on agar plates.

The isolated Fab fragment G8 that showed specificity for the HLA-A1/MAGE-A1 complex was of low affinity (250 nM). Therefore, the selected TCR-like Ab, Fab-G8, which is highly specific for the peptide melanoma-associated Ag-A1 presented by the HLA-A1 molecule was affinity matured via a combination of L chain shuffling, H chain-targeted mutagenesis, and in vitro selection of phage display libraries, essentially as described (Chames P, et al. TCR-Like Human Antibodies Expressed on Human CTLs Mediate Antibody Affinity-Dependent Cytolytic Activity, The Journal of Immunology, 2002, 169: 1110-1118). This procedure yielded a Fab-G8 Ab derivative, Fab-Hyb3, with an 18-fold improved affinity (14 nM) yet identical peptide fine specificity.

In brief:

Chain-shuffling library construction. To build a L chain-shuffling (LS) library, the G8 V_{H} gene was cloned into a vector containing a library of human Ab κ and λL chains. The latter libraries were generated during the construction of the large nonimmune Fab library. Briefly, the pCES1 vector containing Fab-G8 was digested with SfiI and BstEII, and the fragment corresponding to G8 V_{H} was gel purified and extracted using the QiaEX method (Qiagen, Valencia, Calif.). The κ and λ libraries were similarly digested and gel purified. Large-scale ligations (using 20 μg of insert and 20 μg of vector) were performed overnight at 16° C.; the mixture was ethanol precipitated and introduced into Escherichia coli TG1 cells by electroporation. Cells were plated on 2×TY agar plates containing 100 μg/ml ampicillin and 2% glucose. After overnight incubation at 30° C., cells were scraped from the plates and stored at −80° C. in 2×TY containing 15% glycerol.

H chain CDR3 mutagenesis for H chain-CDR3 spiking (HS) library construction. To create the HS library in a one-step PCR amplification of the V_{H} gene, we introduced diversity in the 13 amino acid residues of the H chain CDR3 by using a primer hybridizing on the CDR3 plus FR4 region. The primer used was 5′-GCTTGAGACGGTGACCGTGGTCCCTTGGCCCCAGACGTCCATAC CGTAATAGTAGTAGTGGAAACCACCACCCCTCGCACAGTAATACACAGCC-3′, with the underlined residues using 90% of the wild-type nucleotide and 10% of an equimolar mix of A, T, C, and G (purchased from Eurogentec, Liege, Belgium). The V_{H} fragment was amplified by PCR using the pCES1-Fab-G8 as template. This fragment was digested by SfiI and BstEII and cloned into the pCES1 vector containing the G8 L chain. A library was made as before. Fingerprinting analysis was performed using the primers pUC reverse (5′-AGCGGATAACAATTTCACACAGG-3′) and fd-tet-seq24 (5′-TTTGTCGTCTTTCCAGACGTTAGT-3′). DNA sequencing was performed by Eurogentec using pUC reverse for V_{L} and CH1-fw (5′-GAAGTAGTCCTTGACCAGGC-3′) for V_{H} Finally, The Fab Hyb3 was obtained by combining the variable light chain from the best HLA-A1/MAGE-A1 binder obtained from the chain shuffling library, and the variable heavy chain derived from the H-chain library.

Construction of the Hvb3 scFv

A single chain Fv fragment of the Fab Hyb3 was generated in two steps. First, the genes encoding the Fab Hyb3 Heavy and Light chain fragments were subjected to PCR (primers sequences underlined in sequence of scFv Hyb3) to introduce restriction sites that allow gene insertion into the pBlue-212 vector (Willemsen, R. A. et al. Grafting primary human T lymphocytes with cancer-specific chimeric single chain and two chain TCR. Gene Ther. 7: 1369-1377). The sequence of the resulting scFv was verified and introduced into a retroviral expression cassette for analysis of TCR-like specificity, essentially as described (Ralph A. et al. T Cell Retargeting with MHC Class I-Restricted Antibodies: The CD28 Costimulatory Domain Enhances Antigen-Specific Cytotoxicity and Cytokine Production, The Journal of Immunology, 2005, 174: 7853-7858). This retroviral expression cassette, termed pBullet-cassette, contains: (1) the G250 variable heavy chain signal sequence, and (2) a constant kappa chain linker (Cκ), the CD4 transmembrane domain and the intracellular γ chain (CD4/γ). All domains were derived from the G250 specific chimeric scFv-HKCD4/γ receptor nucleic acid construct (Weijtens, et al. Gene Therapy. 1998, 9:1195-203). The scFv Hyb3 fragment was inserted in Sfi I and Not I digested pBullet cassette DNA, linking the scFv Hyb3 5′ to the signal sequence and 3′ to the CD4/γ fragment in the pBullet vector. Specific binding of the scFv was confirmed by: 1) introduction into primary human peripheral blood lymphocytes, 2) analysis of lymphocyte reactivity towards relevant and irrelevant human melanoma cells, as described in Willemsen, et al. “T Cell Retargeting with MHC Class I-Restricted Antibodies: The CD28 Costimulatory Domain Enhances Antigen-Specific Cytotoxicity and Cytokine Production”, The Journal of Immunology, 2005, 174: 7853-7858).

| Sequence of the scFv Hyb 3 | |
|---|---|
| 1 | GCGGCCCAGCCGGCCATGGCCGAGGTGCAGCTGGTGGAGTCTGGGGGAGGCTTGGTACAG |
| 61 | CCTGGCAGGTCCCTGAGACTCTCCTGTGCAGCCTCTGGATTCACCTTTGATGATTATGCC |
| 121 | ATGCACTGGGTCCGGCAAGCTCCAGGGAAGGGCCTGGAGTGGGTCTCAGGTATTAGTTGG |
| 181 | AATAGTGGTAGCATAGGCTATGCGGACTCTGTGAAGGGCCGATTCACCATCTCCAGAGAC |
| 241 | AACGCCAAGAACTCCCTGTATCTGCAAATGAACAGTCTGAGAGCTGAGGACACGGCTGTG |
| 301 | TATTACTGTGCGAGGGGTCGTGGATTCCACTACTACTATTACGGTATGGACATCTGGGGC |
| 361 | CAAGGGACCACGGTCACCGTCTCAAGATCTGGCTCTACTTCCGGTAGCGGCAAATCCTCT |
| 421 | GAAGGCAAAGGTACTAGACAGTCTGTGCTGACTCAGCCACCCTCGGTGTCAGTGGCCCCA |
| 481 | GGACAGACGGCCAGGATTACCTGTGGGGGAAACAACATTGGAAGTAGAAGTGTGCACTGG |
| 541 | TACCAGCAGAAGCCAGGCCAGGCCCCTGTGCTGGTCGTCTATGATGATAGCGACCGGCCC |
| 601 | TCAGGGATCCCTGAGCGATTCTCTGGCTCCAACTCTGGGAACATGGCCACCCTGACCATC |
| 661 | AGCAGGGTCGAAGCCGGGGATGAGGCCGACTATTACTGTCAGGTGTGGGATAGTCGTACT |
| 721 | GATCATTGGGTGTTCGGCGGAGGGACCAAGCTGACCGTCCTCGCGGCCGC |

1.2: Construction of a Single Chain T Cell Receptor with HLA-A2 Restricted, gp100 Antigen Specificity.

A single chain T cell receptor (scTCR) with HLA-A2/gp100 specificity was constructed from the cytolytic T cell clone MPD essentially as described for a scTCR with HLA-A1/MAGE-A1 specificity (R A Willemsen, et al. Grafting primary human T lymphocytes with cancer-specific chimeric single chain and two chain TCR. Gene Therapy. 2000, 7:1369-77).

First, The TCR αβ gene usage of CTL MPD was determined by TCR family typing PCR, as described (Schaft N. Peptide fine specificity of anti-glycoprotein 100 CTL is preserved following transfer of engineered TCR alpha beta genes into primary human T lymphocytes. J Immunol. 2003 170:2186-94). Sequence analysis of the obtained fragments then allowed the design of primers to specifically amplify the TCR alpha variable region and the TCR beta variable and constant regions (primer sequences underlined in scTCR-MPD sequence). These TCR fragments were then cloned into the vector pBluescript-linker essentially as described for the scFv Hyb3 to obtain a scTCR Vα-linker-VβCβ. The linker sequence of the resulting scTCR is underlined in the sequence of scTCR MPD.

To verify specific binding of the scTCR-MPD to HLA-A2/gp100 the scTCR Vα-linker-VβαCβ DNA was first cloned into the pBullet cassette as described for the scFv Hyb3, and analysed for functional binding to HLA-A2/gp100 positive tumor cells after introduction into primary human peripheral blood lymphocytes, as described for scFv Hyb3.

| Sequence of the scTCR MPD | |
|---|---|
| 1 | GGCCCAGCCGGCCATGGCCCAACAACCAGTGCAGAGTCCTCAAGCCGTGGTCCTCCGAGA |
| 61 | AGGGGAAGATGCTGTCATCAACTGCAGTTCCTCCAAGGCTTTATATTCTGTACACTGGTA |
| 121 | CAGGCAGAAGCATGGTGAAGCACCCGTCTTCCTGATGATATTACTGAAGGGTGGAGAACA |
| 181 | GAAGGGTCATGACAAAATATCTGCTTCATTTAATGAAAAAAAGCAGCAAAGCTCCCTGTA |
| 241 | CCTTACGGCCTCCCAGCTCAGTTACTCAGGAACCTACTTCTGTGGCACAGAGACGAACAC |
| 301 | CGGTAACCAGTTCTATTTTGGGACAGGGACAAGTTTGACGGTCATTCCAGGATCTGGCTC |
| 361 | TACTTCCGGTAGCGGCAAATCCTCTGAAGGCAAAGGTACTAGAGGAGATGCTGGAGTTAT |
| 421 | CCAGTCACCCCGGCACGAGGTGACAGAGATGGGACAAGAAGTGACTCTGAGATGTAAACC |
| 481 | AATTTCAGGACACGACTACCTTTTCTGGTACAGACAGACCATGATGCGGGGACTGGAGTT |
| 541 | GCTCATTTACTTTAACAACAACGTTCCGATAGATGATTCAGGGATGCCCGAGGATCGATT |
| 601 | CTCAGCTAAGATGCCTAATGCATCATTCTCCACTCTGAAGATCCAGCCCTCAGAACCCAG |
| 661 | GGACTCAGCTGTGTACTTCTGTGCCAGCAGTTTGGGGCGGTACAATGAGCAGTTCTTCGG |
| 721 | GCCAGGGACACGGCTCACCGTGCTAGAGGACCTGAAAAACGTGTTCCCACCCGAGGTCGC |
| 781 | TGTGTTTGAGCCATCAGAAGCAGAGATCTCCCACACCCAAAAGGCCACACTGGTATGCCT |
| 841 | GGCCACAGGCTTCTACCCCGACCACGTGGAGCTGAGCTGGTGGGTGAATGGGAAGGAGGT |
| 901 | GCACAGTGGGGTCAGCACAGACCCGCAGCCCCTCAAGGAGCAGCCCGCCCTCAATGACTC |
| 961 | CAGATACTGCCTGAGCAGCCGCCTGAGGGTCTCGGCCACCTTCTGGCAGAACCCCCGCAA |
| 1021 | CCACTTCCGCTGTCAAGTCCAGTTCTACGGGCTCTCGGAGAATGACGAGTGGACCCAGGA |
| 1081 | TAGGGCCAAACCTGTTCACCCAGATCGTCAGCGCCGAGGCCTGGGGTAGAGCAGACGCGGC |
| 1141 | CGC |

### 2.: Construction of scFv-BIX and scTCR-BIX

This section describes the construction of a nucleic acid encoding a TCR-base polypeptide provided with the binding ligand BTX (scTCR-BTX) and nucleic acid encoding an antibody-based polypeptide provided with the binding ligand BTX (scFv-BTX).

The genes encoding scFv Hyb3 and scTCR-MPD (see 1.1 and 1.2) were linked to the alpha-bungarotoxin (BTX) gene (sequence with restriction sites below) that was generated as an synthetic gene by Baseclear b.v. (Leiden, the Netherlands) and cloned into the pGEM11 vector. The BTX gene was cloned into the Not I and Xho I digested pBullet Hyb3-CD4/γ and pBullet MPD-CD4/γ, removing the CD4/γ fragment and linking the BTX protein 5′ to the scFv and scTCR. This resulted in the vectors pBullet-scFv Hyb3/BTX and pBullet-scTCR MPD/BTX.

| Sequence of alpha-bungarotoxin | |
|---|---|
| 1 | GCGGCCGCTATCGTATGCCACACAACAGCTACTTCGCCTATTAGCGCTGTGACTTGTCCA |
| 61 | CCTGGGGAGAACCTATGCTATAGAAAGATGTGGTGTGATGTATTCTGTTCCAGCAGAGGA |
| 121 | AAGGTAGTCGAATTGGGGTGTGCTGCTACTTGCCCTTCAAAGAAGCCCTATGAGGAAGTT |
| 181 | ACCTGTTGCTCAACAGACAAGTGCAACCCACATCCGAAACAGAGACCTGGTTGACTCGAG |

Amino acid composition of the alpha bungarotoxin gene
A A A I V C H T T A T S P I S A V T C P P G E N L C Y R K M W C
D V F C S S R G K V V E L G C A A T C P S K K P Y E E V T C C S
T D K C N P H P K Q R P G

### 3: Construction of TR1-tag and Hexa-tag

In order to be able to generate trimeric and hexameric multivalent protein complexes comprising scfv Hyb3 or scTCR MPD polypeptides, two distinct linker peptides were generated by PCR technology. The TRI-tag linker peptide contains a trimerization motif and one BTX binding site such that trimerized TRI-tag linker peptides have three high affinity binding sites for polypeptides provided with the binding ligand BTX. The Hexa-tag linker peptide contains a trimerization motif and two BTX binding sites for a BTX-polypeptide, such that trimerized Hexa-tag linker peptides have in total six binding sites for BTX-containing polypeptides.

### 3.1: Construction of TR1-tag Linker Peptide.

Synthetic gene fragments encoding a trimerisation motif as well as a binding site for BTX-polypeptide were generated by PCR. Oligonucleotides encoding the neck region peptide (NRP) of human lung surfactant protein D as well as the BTX binding site sequences as well as the complementary sequence were generated and used as a template in PCR to generate a synthetic gene that encodes: 1) the signal sequence of the interleukin 2 protein, 2) the NRP sequence and 3) the BTX binding site. The resulting nucleic acid construct (TRI-tag) was verified by sequence analysis (see below for sequence) and cloned into the retroviral vector pBullet using Nco I and Xho I restriction sites.

| Sequence of TRI-tag | |
|---|---|
| 1 | CCATGGACAGGATGCAACTCCTGTCTTGCATTGCACTAAGTCTTGCACTTGTCACTCCTG |
| 61 | ACGTAGCAAGCTTACGACAACAGGTAGAAGCCTTGCAAGGGCAGGTACAACACTTACAGG |
| 121 | CGGCATTTAGCCAATACAAAAAGGTAGAGTTGTTTCCAAACTGGCGGTACTACGAGAGCA |
| 181 | GCCTGGAGCCCTACCCCGACTAACTCGAG |

```
Amino-acid composition of TRI-tag
M D R M Q L L S C I A L S L A L V T P D V A S L R Q Q V E A L Q G
                      IL-2 signal sequence Trimerization motif
Q V Q H L Q A A F S Q Y K K V E L F P N W R Y Y E S S L E P Y P D
                                                                                      BTX binding site
```

### 3.2. Construction of Hexa-tag Linker Peptide

A nucleic acid encoding a linker peptide that allows for the production of a protein complex that comprises six TCR-(like) polypeptides was constructed. This involved the introduction by PCR of a second BTX binding site sequence in the TRI-tag peptide (see item 3.1) in between the IL-2 signal sequence and the trimerization motif. This resulted in the following nucleic acid construct:

| Sequence of Hexa-tag | |
|---|---|
| 1 | CCATGGACAGGATGCAACTCCTGTCTTGCATTGCACTAAGTCTTGCACTTGTCACTTGGC |
| 61 | GGTACTACGAGAGCAGCCTGGAGCCCTACCCCGACCCTGACGTAGCAAGCTTACGACAAC |
| 121 | AGGTAGAAGCCTTGCAAGGGCAGGTACAACACTTACAGGCGGCATTTAGCCAATACAAAA |
| 181 | AGGTAGAGTTGTTTCCAAACGGATGGCGGTACTACGAGAGCAGCCTGGAGCCCTACCCCG |
| 241 | ACTAACTCGAG |

```
Amino-acid composition of HEXA-tag
M D R M Q L L S C I A L S L A L V T W R Y Y E S S L E P Y P D P D V A S L R
                      IL-2 signal sequence BTX binding site
Q Q V E A L Q G Q V Q H L Q A A F S Q Y K K V E L F P N G W R Y Y E S S L E
                            Trimerization motif BTX binding site
P Y P D
```

Furthermore, a HEXA-tag linker peptide construct was prepared which not only contains two BTX binding site sequences, but also His-tag and c-myc-tag sequences that allow for protein purification, separated by short flexible linker sequences (for sequence see below).

| Sequence of HEXA-tag with 6 × His and c-myc tag | |
|---|---|
| 1 | CCATGGACAGGATGCAACTCCTGTCTTGCATTGCACTAAGTCTTGCACTTGTCACTTGGC |
| 61 | GGTACTACGAGAGCAGCCTGGAGCCCTACCCCGACGGATCTGGATCTGGCTCTGGATCTG |
| 121 | AACAAAAACTTATTTCTGAAGAAGATCTGGGATCTGGCTCTGGATCTGGCTCTCCTGACG |
| 181 | TAGCAAGCTTACGACAACAGGTAGAAGCCTTGCAAGGGCAGGTACAACACTTACAGGCGG |
| 241 | CATTTAGCCAATACAAAAAGGTAGAGTTGTTTCCAAACGGAGGATCTGGATCTGGCTCTG |
| 301 | GATCTCATCATCATCACCATCACGGAGGATCTGGATCTGGCTCTGGATCTTGGCGGTACT |
| 361 | ACGAGAGCAGCCTGGAGCCCTACCCCGACTAACTCGAG |

```
Amino-acid composition of HEXA-tag with 6 × His and c-myc tag
M D R M Q L L S C I A L S L A L V T W R Y Y E S S L E P Y P D G S G S G S G
IL-2 signal sequence BTX binding site
                linker
SE Q K L I S E E D L G S G S G S G S P D V A S L R Q Q V E L Q G Q V Q H
            c-myc tag linker NRP trimerization motif
L Q A A F S Q Y K K V E L F P N G G S G S G S G S H H H H H H G G S G S G S
                                                                    linker 6 × His tag linker
G S W R Y Y E S S L E P Y P D
                  BTX binding site
```

### 4.: Production of Trimeric and Hexameric scFv-Hyb3/BTX and scTCR-MPD/BTX Protein Complexes

Trimeric and hexameric scFv-Hyb3/BTX and scTCR-MPD/BTX protein complexes called Tri-TAG-scFv/scTCR and hexa-TAG-scFv/scTCR, respectively were produced in human HEK-293T cells (293T). To this end, the pBullet vector encoding the polypeptides and linker peptides for either the trimeric or hexameric complex were introduced into 293T cells by calcium phosphate transfection using the Cellfect kit from Amersham bioscience. One day after the transfection the tissue culture medium of the transfected cells was replaced with fresh medium and the cells were allowed to produce the proteins for 3 to 5 days. Because of the IL-2 signal sequence the linker peptides were secreted into the culture medium. At day 3 or 5 the medium containing the assembles complexes was harvested, passed through a 0.22 μM filter, and used immediately for induction of apoptosis, or stored at −80° C.

4.1: Induction of Apoptosis Using Hexameric scFvHyb3 Protein

To test the capacity of trimeric and hexameric scFv Hyb3 to induce apoptosis of HIA-A1^{POS}, MAGE-A1^{POS} melanoma cells, 1×10⁶ MZ2-MEL 3.0 cells in 6 well plates were incubated for 4 hours with 3 ml of tissue culture supernatant obtained from cells that were transfected with the DNA constructs encoding the Tri-tag or Hexa-tag, together with constructs encoding scFv Hyb3. After 4 hours, cells were 1) washed with PBS, 2) harvested by trypsinisation, followed by washing in medium 3) washed with PBS, and finally stained with the caspase-3 substrate FAM-VAD-FMC according the instructions of the manufacturer.

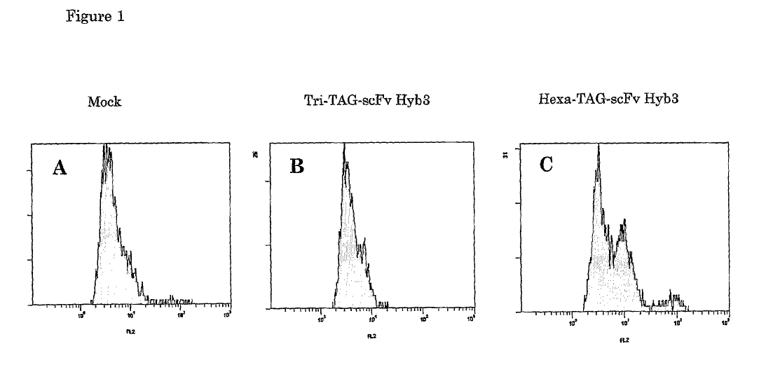

FIG. 1 demonstrates that Caspase-3 activity can only be detected in MZ2-MEL3.0 cells that have been incubated with the supernatant containing the hexameric scFv Hyb3 protein, and not when incubated with tissue culture media that contains either trimeric scFv Hyb3 protein or tissue culture medium that has been obtained from 293T cells that were transfected with the empty pBullet vector. This indicates that the hexameric but not the trimeric multivalent monospecific complex induces apoptosis in human cells expressing the tumour antigen MAGE-A1.

### 4.2: HLA-A1/MAGE-A1 Specific Induction of Apoptosis by Hexa-TAG-scFv Hyb3.

To test the specificity of apoptosis induction e.g. the HLA-A1/MAGE-A1 restricted specificity, supernatant containing Hexa-TAG-Hyb 3 was incubated with monolayers of either the HLA-A1^{POS}, MAGE-A1^{POS} melanoma cell line MZ2-MEL 3.0 and or the mutant MZ2-MEL2.2 melanoma cell line which has lost MAGE-A1 antigen. To this end, 1×10⁶ melanoma cells were incubated for 4 hours with: 1) supernatant derived from mock transfected 293T cells (empty pBullet vector), 2) supernatant derived from 293T cells transfected with the irrelevant construct pBullet scTCR MPD together with the pBullet vector Hexa-tag, or 3) supernatant derived from 293T cells transfected with the pBullet scFv Hyb3 vector together with the pBullet Hexa-tag vector. Induction of apoptosis was analysed by double staining with both Annexin V (PS exposure) and 7-AAD (viability dye).

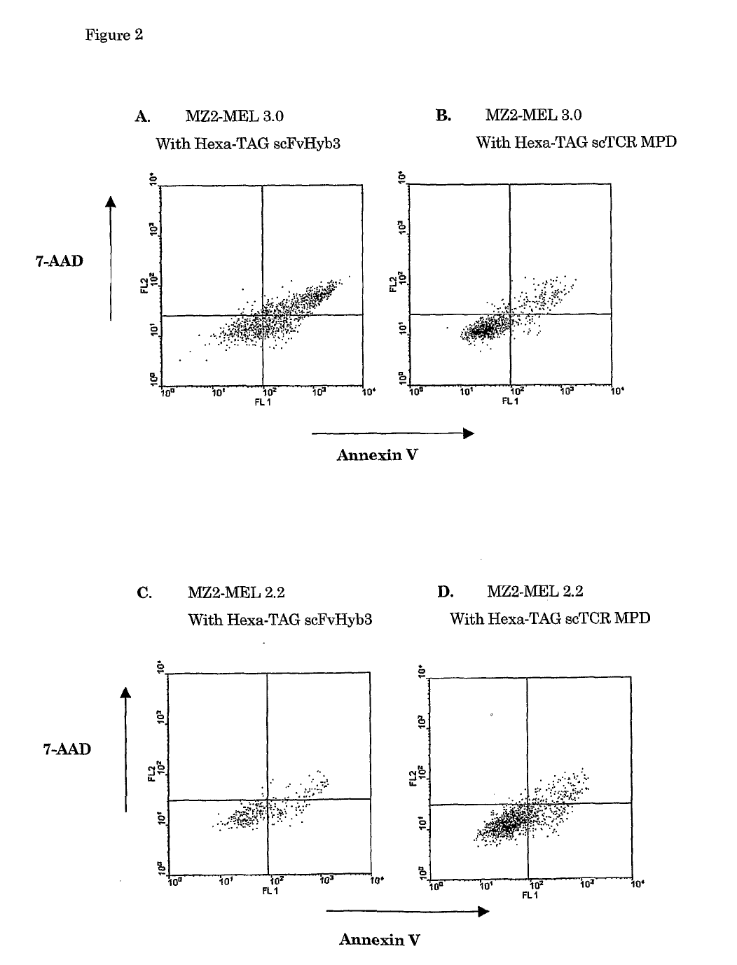

As shown in FIG. 2, only the HLA-A1^{POS}, MAGE-A1^{POS} melanoma cell line MZ2-MEL 3.0 showed a significant induction of apoptosis, measured by Annexin V and 7-AAD staining, after incubation with supernatant containing Hexa-TAG scFv Hyb3 protein (FIG. 2A). In contrast, none of the other conditions showed any sign of apoptosis above background levels. MZ2-MEL 3.0 cells incubated with the irrelevant Hexa-TAG scTCR MPD are shown as an example (FIG. 2B). The antigen-specificity of apoptosis induction by the protein complex was demonstrated by the fact that the viability of the MAGE-A1 antigen lost mutant cell line MZ2-MEL2.2, which was derived from the MAGE-A1 antigen positive cell line MZ2-MEL 3.0 was not affected by the Hexa-TAG-scFv Hyb3 protein (FIG. 2C, D).

4.3: HLA-A2/gp100 Specific Induction of Apoptosis by Hexa-TAG scTCR MPD

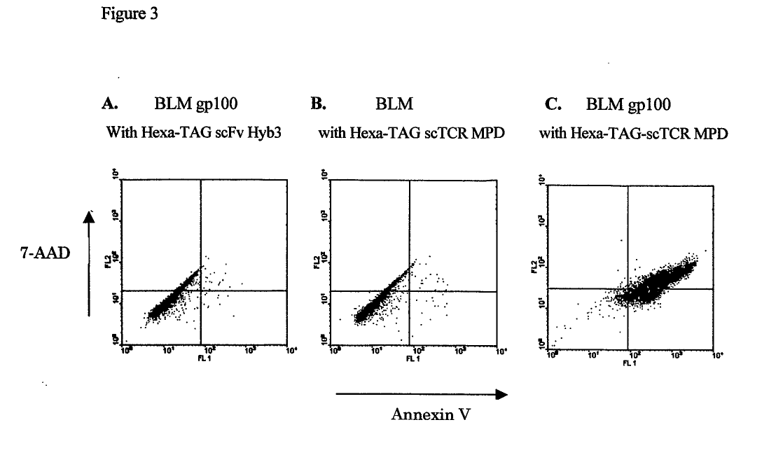

The gp100 antigen is highly expressed in melanocytic cells. HLA-A2/gp100 specific induction of apoptosis by the Hexa-TAG scTCR MPD protein complex was analysed by incubation of HLA-A2^{POS} BLM and HLA-A2/gp100^{POS} BLM-gp100 melanoma cells with tissue culture supernatant obtained by transfection of 293T cells with the pBullet Hexa-tag vector together with the pBullet scTCR MPD/BTX vector. 4 hours after incubation, cells were harvested and stained with Annexin V and 7-AAD to determine the induction of apoptosis. FIG. 3 demonstrates that apoptosis is only induced in gp100-positive melanoma cells (FIG. 3C), and not in gp100-negative cells (FIG. 3B). Furthermore, apoptosis of gp100 positive melanoma cells is not induced by irrelevant Hexa-TAG scFv Hyb3 proteins (FIG. 3A). These data demonstrate that the killing of target cells is antigen-specific.

1. A multivalent monospecific protein complex comprising at least six polypeptides capable of recognizing and binding to a specific Major Histocompatibility Complex (MHC)-peptide complex.

2. Protein complex according to claim 1, further comprising at least one linker peptide comprising a multimerisation motif and at least one binding site for one of said at least six polypeptides comprising a binding ligand for said binding site.

3. Protein complex according to claim 2, wherein the binding site is the alpha-bungarotoxin (BTX) binding site and wherein the ligand is BTX.

4. Protein complex according to claim 2, wherein said multimerisation motif is a mammalian, preferably human, multimerisation motif.

5. Protein complex according to claim 2, wherein said linker peptide comprises a trimerisation motif and at least two binding sites for at least two of said at least polypeptides.

6. Protein complex according to claim 5, wherein said trimerisation motif is selected from the group consisting of the Neck Region Peptide (NRP) of human Lung Surfactant D protein, the trimerisation signal of bacteriophage T4 fibritin and the modified Zipper motif.

7. Protein complex according to claim 6, wherein said trimerisation motif is NRP of human Lung Surfactant D protein.

8. Protein complex according to claim 1, wherein at least one polypeptide comprises amino acid sequences corresponding to extracellular constant (C) and variable (V) region sequences of a native TCR.

9. Protein complex according to claim 8, wherein at least one polypeptide is a single-chain T cell receptor (scTCR) polypeptide.

10. Protein complex according to claim 8, wherein at least one polypeptide is a two-chain TCR (tcTCR) comprising the extracellular variable (V) and constant (C) regions of an antigen-specific TCR.

11. Protein complex according to claim 9, wherein said scTCR or tcTCR comprise the alpha and beta chains pair or the gamma and delta chain pair of an antigen-specific TCR.

12. Protein complex according to claim 1, wherein at least one polypeptide is an antigen-specific MCH-restricted antibody or a functional fragment thereof, preferably a single chain variable antibody fragment (scFv).

13. Protein complex according to claim 1, wherein said at least six polypeptides are capable of recognizing and binding to a viral epitope, a cancer-specific epitope or an epitope associated with autoimmune disorders.

14. Protein complex according to claim 13, wherein said epitope is selected from the group consisting of HTLV-I epitopes, HIV epitopes, EBV epitopes, CMV epitopes, melanoma epitopes.

15. A method for providing a protein complex according to claim 1, comprising the steps of:
providing a host cell with one or more nucleic acid(s) encoding said at least six polypeptides capable of recognizing and binding to a specific Major Histocompatibility Complex (MHC)-peptide complex and, optionally, a linker peptide;
allowing the expression of said nucleic acids by said host cell; and
allowing the assembly of the resulting peptides into the protein complex.

16. Method according to claim 15, wherein providing said nucleic acid(s) encoding said polypeptides comprises isolating and cloning a scFv from a phage display library.

17. Method according to claim 15, wherein the expressed peptides are secreted and assembled in the culture medium of said host cell.

18. A method for inducing ex vivo or in vivo apoptosis of a target cell, comprising contacting said cell with a protein complex according to claim 1.

19. Method according to claim 18, comprising producing the components of said protein complex wherein the expressed peptides are secreted and assembled in the culture medium of said host cell and contacting said target cell with the culture medium comprising the protein complex.

20. Protein complex according to claim 1 as medicament.

21. Protein complex according to claim 20 as antiviral or anti-tumour agent.

22. The use of a protein complex according to claim 1 for the manufacture of a medicament for the treatment of cancer, an autoimmune disease or a viral or microbial infection.

23. Use according to claim 22, wherein the cancer is melanoma.

24. A pharmaceutical composition comprising a protein complex according to claim 1 and a pharmaceutically acceptable carrier.

25. Linker peptide comprising a multimerisation motif, preferably a human multimerisation motif, more preferably a human trimerisation motif, and at least two binding sites, optionally furthermore comprising at least one affinity tag that allows for the purification of said linker peptide.

26. Nucleic acid encoding a linker peptide according to claim 25.
